(19) United States
(12) Patent Application Publication
(10) Pub. No.: US 2010/0144781 A1
(43) Pub. Date: Jun. 10, 2010

(54) METHODS OF TREATING PSYCHOSIS AND SCHIZOPHRENIA BASED ON POLYMORPHISMS IN THE ERBB4 GENE

(76) Inventors: Dong-Jing Fu, Flemington, NJ (US); Qingqin S. Li, Flemington, NJ (US); Dai Wang, Bridgewater, NJ (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 12/608,386

(22) Filed: Oct. 29, 2009

Related U.S. Application Data
(60) Provisional application No. 61/109,311, filed on Oct. 29, 2008.

(51) Int. Cl. A61K 31/4375; C12Q 1/68

(52) U.S. Cl. 514/300; 435/6

(57) ABSTRACT

The present application is directed to the use of genetic polymorphism in the ErbB4 gene to predict whether a patient is likely to respond to psychotic medication Paliperidone. The polymorphism in the ErbB4 gene is also used to predict whether a patient is likely to display placebo effect among patients in need of psychotic treatment. A method of treating patients with antipsychotic medication Paliperidone using the polymorphism in the ErbB4 gene and a kit of are also provided.

### CROSS-REFERENCE TO RELATED APPLICATIONS

This application claims the benefit of U.S. Provisional Application No. 61/109,311, filed Oct. 29, 2008, the entire disclosure of which is hereby incorporated in its entirety.

### FIELD OF THE INVENTION

The present application provides methods to treat or diagnose psychotic conditions including schizophrenia and related disorders. In particular, this application is directed to the use of polymorphism analysis to determine whether a patient is likely to display a placebo response or respond to treatment with Paliperidone, and methods to determine optimal treatment strategies.

### BACKGROUND

Psychoses exert a tremendous emotional and economic toll on the patients, their families, and society as a whole. Psychotic conditions, such as schizophrenia and related disorders (e.g. schizoaffective disorder), and affective disorders (mood disorders) with psychotic symptoms (e.g. Bipolar Disorder) are complex and heterogeneous diseases of uncertain aetiology.

Schizophrenia is a severe psychotic or neuropsychiatric disorder affecting about 1% of the general population. Schizophrenia is characterized as having both positive symptoms including hallucinations, delusions, and conceptual disorganization, and negative symptoms including social withdrawal, blunted affect, and poverty of speech. Clinical rating scales such as the Positive and Negative Syndrome Scale (PANSS) (Kay et al., Compr Psychiatry, 1991, 32: 355-361), the Scale for the Assessment of Negative Symptoms (SANS) (Andreasen et al., Arch Gen Psychiatry, 1982, 39: 784-788), and the Scale for the Assessment of Positive symptoms (SAPS) (Andreasen 1984. Scale for the assessment of positive symptoms. Iowa City, Iowa: University of Iowa) provide criteria to differentiate and rate positive and negative symptoms.

Early studies have shown that abnormal activity of the neurotransmitter dopamine is a hallmark of schizophrenia. Reduced dopaminergic activity in the mesocortical system results in negative symptoms and enhanced dopaminergic activity in the mesolimbic system results in positive or psychotic symptoms. Patients showing schizophrenia and other psychotic symptoms are usually treated with classical antipsychotic drugs, the neuroleptics, which block central dopamine receptors. The ability of these drugs to antagonize dopamine D2 receptors correlates with antipsychotic efficacy. The neuroleptic drugs include chlorpromazine, thioridazine, fluphenazine, haloperidol, flupenthixol, molindone, loxapine, and pimozide. Though the neuroleptics are effective in treating the positive symptoms of schizophrenia, they have little or no effect on negative symptoms. Further, neuroleptics cause extrapyramidal symptoms, including rigidity, tremor, bradykinesia (slow movement), and bradyphrenia (slow thought), as well as tardive dyskinesias and dystonias.

Recently, several other neurotransmitters, including serotonin, glutamate, and GABA, have also been shown to be involved in schizophrenia conditions. For example, in humans, reduced glutamatergic transmission mimics schizophrenia symptoms whereas enhanced glutamatergic transmission alleviates schizophrenia symptoms. This leads to the development of the second generation atypical antipsychotic drugs. The atypical antipsychotic drugs are a different class of antipsychotic drugs which have different receptor binding profile and effectiveness against the symptoms of schizophrenia. Most atypical antipsychotics bind central serotonin 5-HT2 receptors in addition to dopamine D2 receptors. Unlike the neuroleptics, atypical antipsychotics improve negative as well as positive symptoms. In addition, they cause minimal extrapyramidal symptoms and rarely cause tardive dyskinesias, akathisia, or acute dystonic reactions which associated with the neuroleptics therapy. The efficacy of atypical antipsychotic drugs in improving over all schizophrenia symptoms has been correlated to their capability to modulate additional neurotransmission pathways. The atypical antipsychotics are effective for the treatment of schizophrenia and have been used as a first-line medication for neuroleptic resistant patients. Certain side effects have been reported in atypical antipsychotic therapy; for example, the use of clozapine may cause severe blood disorder (agranulocytosis), weight gain, and diabetics. In addition to biochemical and neurological factors, genetic components have been mapped with schizophrenia and other psychotic conditions. The genome-wide linkage studies have associated chromosomal structure abnormalities or copy number variants on chromosomes 22q, 13q, 6p, 8p, 1q, 15q, and 2q with schizophrenia and bipolar disorder patients. For example, rare chromosomal deletions and duplications at 1q21.1 and 15q13.3 have been reported to increase risk of schizophrenia (The International Schizophrenia Consortium Nature 2008, 455, 237-241, Stefansson et al. Nature 2008, 455: 232-236, Xu et al. Nature Genetics 2008, 40: 880-885, and Walsh et al. Science 2008, 320:539-543). In addition, detailed mapping studies on these chromosome regions have identified Catechol-o-methyltransferase (COMT), D-amino acid oxidase (DAO), Disrupted-in-schizophrenia (DISC)-1, Dysbindin (DTNBP1), and Neuregulin 1 (NRG1) as potential susceptibility genes. Other studies have examined functional candidate genes, including dopamine transporter, serotonin 5-HT receptor, N-methyl-D-aspartate (NMDA) receptor, glutamate transporter SLC1A, and G-protein coupled receptors, and their roles in schizophrenia conditions. These studies indicate the complexity of schizophrenia and psychotic conditions and suggest several genes or gene clusters contribute to the disease susceptibility.

Some of these genetic components have been used to predict schizophrenia conditions, include COMT, dopamine transporter, 5-HT2A, 5-HT2C, NMAD receptor, NRG1, DISC-1, and DAO (See reviews by Sawa and Snyder, Molecular Medicine, 2003: 3-9; Owen et al., Trends in Genetics, 2005, 21:518-525; Craddock et al., Journal of Medical Genetics, 2005, 42:193-204; Lang et al, Cellular Physiology Biochemistry, 2007, 20: 687-702).

Recent development in high-throughput genotyping technology has facilitated genome-wide association studies to explore the pathophysiology of diseases and the patients' responsiveness to treatment at a scale and resolution not feasible previously. The genome-wide association studies have shown potential genetic influence of colony stimulating factor 2 receptor, alpha, low-affinity (granulocyte-macrophage) (CSF2RA), interleukin 3 receptor, alpha (low affinity) (IL3RA), reelin (RELN), coiled-coil domain containing 60 (CCDC60), retinoblastoma-binding protein 1 (RBP1 or ARID4A), and zinc finger protein 804A (ZNF804A) to the susceptibility of schizophrenia (Lencz et al, Molecular Psychiatry 2007, 12: 572-580, Shifman et al, PloS Genetics 2008, 4: e28, Kirov et al, Molecular Psychiatry 2008, Mar. 11 online-publication ahead of print, Sullivan et al, Molecular Psychiatry 2008, 13: 570-584, and O′Donovan et al, Nature Genetics, 2008 Jul. 30, epub ahead of print). The CNTF (ciliary neurotrophic factor), NPAS3 (neuronal PAS domain protein 3), XKR4 (Kell blood group complex subunit-related family, member 4), TNR (tenascin R (restrictin, janusin)), GRIA4 (glutamate receptor, ionotrophic, AMPA 4), GFRA2 (GDNF family receptor alpha 2), NUDT9P1 (nudix (nucleoside diphosphate linked moiety X)-type motif 9 pseudogene 1) genes have been reported to be associated with the responsiveness of schizophrenia patients to the treatment with iloperidone (Lavedan et al Pharmacogenomics 2008, 9(3):289-301 and Lavedan et al Molecular Psychiatry 2008 Jun. 3 epub ahead of print). The CERKL (ceramide kinase-like), SLCO3A1 (solute carrier organic anion transporter family, member 3A1), BRUNOL4 (bruno-like 4, RNA binding protein (Drosophila)), and NRG3 (neuregulin 3) genes were reported to be associated with QT prolongation during treatment (Volpi et al Molecular Psychiatry 2008 Jun. 3 epub ahead of print). US20080027106 discloses the use of genomic analysis to determine a patients' responsiveness to the treatment with iloperidone.

NRG1, mapped to chromosome 8p21-p12, is one of the most supported susceptibility genes for schizophrenia. NRG1 is involved in neuronal migration, differentiation and expression of neurotransmitters acetylcholine, GABA-A, and glutamate. Function of NRG1 is mainly mediated through interaction with the ErbB family of tyrosine kinase receptors. Genetic studies have shown that mutant mice heterozygous for NRG1 or ErbB4 displayed behavior phenotypes of schizophrenia mouse models, which are not observed in knockout ErbB2 or ErbB3 mice (Stefansson et al., American Journal of Human Genetics, 2002, 71:877-892). In addition, genetic studies have shown the association of polymorphisms in NRG1 and ErbB4 genes with schizophrenia (Norton et al., American Journal Medical Genetics B, 2006, 141: 96-101; Silberberg et al., American Journal Medical Genetics B, 2006, 141:142-148; Benzel et al., Behavior Brain Functions, 2007, 3:31; Walsh et al., Science, 2008, 320: 539-543). Further, neurophysiology studies have shown the binding of NRG1-ErbB4 inhibits the N-methyl-D-aspartate (NMDA) receptor and interferes the glutamate transmission at the NMDA receptor (Li et al., Neuron, 2007, 54: 583-597). Combined with previous observations of glutamatergic transmission and schizophrenia symptoms, it is likely that deficiency in the NRG1-ErbB4 signaling pathway results in hypofunction of glutamatergic activity which leads to changes in dopaminergic activity and contributes to schizophrenia susceptibility.

ErbB4, v-erb-a erythroblastic leukemia viral oncogene homolog 4, protein is a member of the tyrosine protein kinase family and the epidermal growth factor receptor subfamily. The ErbB4 protein is well known to be involved with intracellular signaling cascades and induction of cellular responses. Previously, the ErbB4 protein has been targeted for treating or preventing cancer, abnormal cell growth and migration. With the recent understanding of the NRG1/ErbB4 signaling pathway in schizophrenia, modulators on NRG1 and ErbB4 genes have been used as therapeutic targets in treating or preventing schizophrenia as disclosed in WO2008019394, US20060029546, and US20070213264. In addition, the polymorphisms or related genetic information of NRG1 gene have been used for diagnosis of schizophrenia or psychotic conditions as disclosed in US20020094954 and US20050208527.

The treatment and the diagnosis of psychotic disorders with antipsychotic agents have steadily improved over the years. However, there is no reliable means to determine how patients will respond to an antipsychotic agent and what dose level a given patient may require to produce a therapeutic response without severe side effects. Since all antipsychotic agents, even the newer atypical ones, have side effects, this “trial and error” period could be time consuming, unpleasant and even dangerous for the patient and increased the likelihood of non-compliance. Therefore there remains a need for methods to diagnose and treat schizophrenic patients.

### SUMMARY

A marker is provided to predict or diagnose patients' responsiveness to treatment of antipsychotic medication. The marker is a polymorphism present in the ErbB4 gene, specifically the polymorphism of 201 G>A in SEQ ID NO: 1. In one embodiment, a method is provided for predicting likelihood of a patient responding to a treatment with an atypical antipsychotic drug selected from the group consisting of Paliperidone, and pharmaceutically acceptable salts and esters thereof, comprising analyzing nucleotide sequences of two alleles of the ErbB4 gene in the patient; determining a genotype at a polymorphic site at 201 in SEQ ID NO: 1; and identifying the patient with the genotype of GA or GG as being more likely to respond to the drug, and the patient with the genotype of AA as being less likely to respond to the drug.

In another embodiment, a method is provided for predicting whether a patient in need of treatment with antipsychotic drug is likely to display placebo response, comprising analyzing nucleotide sequences of two alleles of the ErbB4 gene in the patient; determining a genotype at a polymorphic site at 201 in SEQ ID NO: 1; and identifying the patient with the genotype of AA as being likely to display placebo response.

In other embodiment, a method for selecting a subject for a clinical study of an antipsychotic medication, comprising analyzing nucleotide sequences of two alleles of the ErbB4 gene in the patient; and determining a genotype at a polymorphic site at 201 in SEQ ID NO: 1; and identifying the subject with the genotype of AA as being likely to display placebo response.

In other embodiment, a method is provided of treating a patient in need of an antipsychotic treatment comprising obtaining a DNA sample from said patient; analyzing nucleotide sequences of the ErbB4 gene in said patient; determining a genotype at a polymorphic site at 201 in SEQ ID NO: 1; and treating said patient with said genotype of GA or GG with an atypical antipsychotic drug selected from the group consisting of Paliperidone, and pharmaceutically acceptable salts and esters thereof. In additional embodiment, a kit is provided for use in determining treatment strategy for a patient with a psychotic disorder comprising a polynucleotide able to recognize and bind to portion of the ErbB4 gene; a container suitable for containing said polynucleotide and a DNA sample from said patient wherein said polynucleotide can contact the ErbB4 gene; and means to detect hybridization of said polynucleotide with the ErbB4 gene.

### DETAILED DESCRIPTION

### ErbB4 Polymorphism

As used herein, the term polymorphism refers to the sequence variation observed in an individual at a polymorphic site. Polymorphisms include nucleotide substitutions, insertions, deletions and microsatellites and may, but need not, result in detectable differences in gene expression or protein function. The term polymorphic site is used to refer to position within a locus at which at least two alternative sequences are found in a population, the most frequent of which has a frequency of no more than 99%. The term single nucleotide polymorphism (SNP) refers to the occurrence of nucleotide variability at a single nucleotide position in the genome, within a population. An SNP may occur within a gene or within intergenic regions of the genome.

A polymorphism or SNP in the ErbB4 gene is provided for predicting likelihood of an individual with a psychotic disorder to treatment with Paliperidone. The polymorphism of 201 G>A in SEQ ID NO: 1, which is referred to as rs6435681 according to the dbSNP database in National Center for Biotechnology Information, is located in the intron 3 region of the ErbB4 gene, which may affect the expression of the ErbB4 gene. Effect(s) of the polymorphism on expression of ErbB4 may be investigated by preparing recombinant cells and/or organisms, preferably recombinant animals, containing a polymorphic variant of the ErbB4 gene. As used herein, the term expression includes, but is not limited to, transcription of the gene into precursor mRNA, splicing and other processing of the precursor mRNA to produce mature mRNA, mRNA stability, translation of the mature mRNA into ErbB4 protein (including codon usage and tRNA availability), and glycosylation and/or other modifications of the translation product, if required for proper expression and function.

The altered level or splicing variant of ErbB4, resulted from polymorphism, may affect normal NRG1-ErbB4 interaction and the schizophrenia development; therefore affect an individual's responsiveness to antipsychotic treatment. The term altered level refers to the level of mRNA or polypeptide expressed from a particular allele, for example the polymorphism at 201 G>A of SEQ ID NO: 1 in the ErbB4 gene, and means the level would lead one of skill in the art to believe that the particular allele is preset. The term splice variant refers to RNA molecules initially transcribed from the same genomic DNA sequence but have undergone alternative RNA splicing. Alternative RNA splicing occurs when a primary RNA transcript undergoes splicing, generally for the removal of introns, which results in the production of more than one mRNA molecule each of which may encode different amino acid sequences. The term splice variant also refers to the proteins encoded by the above RNA molecules produced by alternative splicing.

The SNP of the polymorphic site at 201 of SEQ ID NO: 1 results in three genotypes of GG, GA, and AA. In the clinical population described herein, individuals with the GG or GA genotype at the polymorphic site of 201 in SEQ ID NO: 1 are more likely to respond to the atypical antipsychotic drug Paliperidone; therefore may continue to receive similar dose of the same antipsychotic drug. In addition, individuals with the AA genotype are less likely to respond to the atypical antipsychotic drug Paliperidone; therefore may require a higher dose or an adjunctive medication in addition to Paliperidone, or alternatively, the use of a different atypical antipsychotic drug.

Further, the individuals with the AA genotype at the polymorphism of 201 G>A in SEQ ID NO: 1 are more likely to display placebo response or effect. The term placebo response refers to spontaneous symptom improvement without receiving any medication or treatment for psychotic or psychiatric disorder, and may be as measured by the scales described above. Since patients generally display placebo response within the first 8 weeks, more often, within the first 6 weeks after initial treatment, the knowledge of patients likely to display placebo response greatly facilitates the management of these patients. Alternative treatment strategies, such as frequent supervision or additional antipsychotic drug, are necessary for managing the individual displays placebo response.

It is desirable to predict whether a patient is likely to respond to an atypical antipsychotic drug as appropriate treatment strategies may be applied to effectively manage disease symptoms, minimize side effects, and shorten treatment duration. Also, it is desirable to predict whether a patient is likely to display placebo effect as physicians may apply different treatment strategies to these patients, and clinical trials may be designed by stratifying or selecting appropriate patient populations to maximize the drug efficacy and reduce the cost. Therefore, the assessment of whether a patient is likely to respond to treatment and whether a patient is likely to display placebo response greatly facilitates personalized treatment strategy for disease treatment and maximizes efficacy focus for clinical trials.

In a preferred embodiment, the GA or GG genotype at the polymorphic site of 201 of SEQ ID NO: 1 in the ErbB4 gene is used to predict an individual is more likely to respond to treatment with Paliperidone. In another preferred embodiment, the AA genotype at the same polymorphic site is used to predict an individual is less likely to respond to treatment with Paliperidone. The preferred atypical antipsychotic drug includes Paliperidone, and pharmaceutically acceptable salts and esters thereof, e.g. Paliperidone Palmitate, as disclosed in U.S. Pat. No. 5,254,556 which is incorporated herein by reference in its entirety. In another preferred embodiment, the AA genotype at the polymorphic site of 201 of SEQ ID NO: 1 in the ErbB4 gene is used to predict an individual, in need of psychotic treatment, is likely to display a placebo response.

In addition to the specific polymorphisms disclosed herein, any polymorphism that is in linkage disequilibrium with the polymorphism described above can also serve as additional marker indicating responsiveness to the same drug or therapy as does the SNP that it is in linkage disequilibrium with. Therefore, any SNP in linkage disequilibrium with the SNP disclosed in this application, can be used and is intended to be included herein.

### Detection of Polymorphism, Genotype, and Haplotype

Many different techniques have been used to identify and characterize SNP, such as single-strand conformation polymorphism analysis, heteroduplex analysis by denaturing high-performance liquid chromatography (DHPLC), and computational methods. Due to the wealth of sequence information in public databases, computational tools can be used to identify SNPs in silico by aligning independently submitted sequences for a given gene (either cDNA or genomic sequences).

Other common techniques for assaying SNP or polymorphism include hybridization, sequencing, primer extension, ligase-detection reaction, and cleavage methods. For example, a person skilled in the art may use a cleavage method such as endonucleotide restriction enzyme to cleave DNA fragments for SNP detection. Each of these methods must be connected to an appropriate detection system. Detection technologies include fluorescent polarization, luminometric detection of pyrophosphate release (pyrosequencing), fluorescence resonance energy transfer-based cleavage assays, DHPLC, mass spectrometry, and those disclosed in U.S. Pat. Nos. 6,297,018 and 6,300,063. The disclosures of the above references are incorporated herein by reference in their entirety.

The above methods can also be used for genotyping and/or haplotyping the ErbB4 gene in an individual. As used herein, the terms “ErbB4 genotype” and “ErbB4 haplotype” mean the genotype or haplotype containing the nucleotide pair or nucleotide, respectively, which is present at one or more of the polymorphic sites described herein and may optionally also include the nucleotide pair or nucleotide present at one or more additional polymorphic sites in the ErbB4 gene. The additional polymorphic sites may be currently known polymorphic sites or sites that are subsequently discovered.

In one embodiment, the genotyping method involves isolating from the individual a nucleic acid mixture comprising the two copies of the ErbB4 gene, or a fragment thereof, that are present in the individual, and determining the identity of the nucleotide pair at one or more of the polymorphic sites in the two copies to assign a ErbB4 genotype to the individual. As will be readily understood by the skilled artisan, the two “copies” of a gene in an individual may be the same allele or may be different alleles. In a particularly preferred embodiment, the genotyping method comprises determining the identity of the nucleotide pair at each polymorphic site.

Typically, the nucleic acid mixture or protein is isolated from a biological sample taken from the individual, such as any body fluid or tissue sample. Any body fluid includes but not limited to, serum, plasma, lymph, cystic fluid, urine, stool, csf, acitic fluid. Suitable tissue samples include whole blood, semen, saliva, tears, urine, fecal material, sweat, buccal smears, skin, and biopsies of specific organ tissues such as muscle or nerve tissue and hair.

In another embodiment, the haplotyping method comprises isolating from the individual a nucleic acid molecule containing only one of the two copies of the ErbB4 gene, or a fragment thereof, that is present in the individual and determining in that copy the identity of the nucleotide at one or more of the polymorphic sites in that copy to assign a ErbB4 haplotype to the individual. The nucleic acid may be isolated using any method capable of separating the two copies of the ErbB4 gene or fragment, including but not limited to, one of the methods described above, with targeted in vivo cloning being the preferred approach. As will be readily appreciated by those skilled in the art, any individual clone will only provide haplotype information on one of the two ErbB4 gene copies present in an individual. If haplotype information is desired for the individual's other copy, additional ErbB4 clones will need to be examined. Typically, at least five clones should be examined to have more than a 90% probability of haplotyping both copies of the ErbB4 gene in an individual. In a preferred embodiment, the nucleotide at each of polymorphic site is identified.

An ErbB4 haplotype pair can be determined for an individual by identifying the phased sequence of nucleotides at one or more of the polymorphic sites in each copy of the ErbB4 gene that is present in the individual. Preferably, the haplotyping method comprises identifying the phased sequence of nucleotides at each polymorphic site in each copy of the ErbB4 gene. When haplotyping both copies of the gene, the identifying step is preferably performed with each copy of the gene being placed in separate containers. However, it is also envisioned that if the two copies are labeled with different tags, or are otherwise separately distinguishable or identifiable, it could be possible in some cases to perform the method in the same container. For example, if first and second copies of the gene are labeled with different first and second fluorescent dyes, respectively, and an allele-specific oligonucleotide labeled with yet a third different fluorescent dye is used to assay the polymorphic site(s), then detecting a combination of the first and third dyes would identify the polymorphism in the first gene copy while detecting a combination of the second and third dyes would identify the polymorphism in the second gene copy.

In both genotyping and haplotyping methods, the identity of a nucleotide (or nucleotide pair) at a polymorphic site(s) may be determined by amplifying a target region(s) containing the polymorphic site(s) directly from one or both copies of the ErbB4 gene, or fragment thereof, and the sequence of the amplified region(s) determined by conventional methods. It will be readily appreciated by the skilled artisan that only one nucleotide will be detected at a polymorphic site in individuals who are homozygous at that site, while two different nucleotides will be detected if the individual is heterozygous for that site. The polymorphism may be identified directly, known as positive-type identification, or by inference, referred to as negative-type identification. For example, where a SNP is known to be guanine and cytosine in a reference population, a site may be positively determined to be either guanine or cytosine for all individual homozygous at that site, or both guanine and cytosine, if the individual is heterozygous at that site. Alternatively, the site may be negatively determined to be not guanine (and thus cytosine/cytosine) or not cytosine (and thus guanine/guanine).

The target region(s) may be amplified using any oligonucleotide-directed amplification method, including but not limited to polymerase chain reaction (PCR) (U.S. Pat. No. 4,965,188), ligase chain reaction (e.g. Barany et al., Proc Natl Acad Sci USA 88:189-193, 1991; WO 90/01069), ligase detection reaction (e.g. Favis et al. 2004, Applications of the Universal DNA Microarray in Molecular Medicine, in Methods in Molecular Medicine: Microarrays in Clinical Diagnostics, T. O. Joos and P. Fortina, Editors. 2004, The Humana Press Inc. USA), oligonucleotide ligation assay (e.g. Landegren et al., Science 241:1077-1080, 1988), transcription-based amplification systems (e.g. U.S. Pat. Nos. 5,130,238; 5,169,766) and isothermal methods (e.g. Walker et al., Proc Natl Acad Sci USA 89:392-396, 1992). Oligonucleotides useful as primers or probes in such methods should specifically hybridize to a region of the nucleic acid that contains or is adjacent to the polymorphic site. Typically, the oligonucleotides are between 10 and 35 nucleotides in length and preferably, between 15 and 30 nucleotides in length. Most preferably, the oligonucleotides are 20 to 25 nucleotides long. The exact length of the oligonucleotide will depend on many factors that are routinely considered and practiced by the skilled artisan.

A polymorphism in the target region may also be assayed before or after amplification using one of several hybridization-based methods known in the art. Typically, allele-specific oligonucleotides are utilized in performing such methods. The allele-specific oligonucleotides may be used as differently labeled probe pairs, with one member of the pair showing a perfect match to one variant of a target sequence and the other member showing a perfect match to a different variant. In some embodiments, more than one polymorphic site may be detected at once using two or more sets of allele-specific oligonucleotides or oligonucleotide pairs. The allele-specific oligonucleotide primer has a 3′ terminal or penultimate nucleotide, which is complementary to only one nucleotide of a particular SNP, thereby acting as a primer for polymerase-mediated extension only if the allele containing that nucleotide is present. Preferably, the members of the set have melting temperatures within 5° C. and more preferably within 2° C., of each other when hybridizing to each of the polymorphic sites being detected. The allele-specific oligonucleotide primers may hybridize to either coding or noncoding strand. The allele-specific oligonucleotide primer for detecting polymorphism in the ErbB4 gene could be developed using techniques known to those of skill in the art.

Hybridization of an allele-specific oligonucleotide or other genotyping oligonucleotide to a target polynucleotide may be performed with both entities in solution or such hybridization may be performed when either the oligonucleotide or the target polynucleotide is covalently or noncovalently affixed to a solid support. Attachment may be mediated, for example, by antibody-antigen interactions, poly-L-Lys, streptavidin or avidin-biotin, salt bridges, hydrophobic interactions, chemical linkages, or UV cross-linking baking Allele-specific oligonucleotides may be synthesized directly on the solid support or attached to the solid support subsequent to synthesis. Solid-supports suitable for use in detection methods of the invention include substrates made of silicon, glass, plastic, paper and the like, which may be formed, for example, into wells (as in 96-well plates), slides, sheets, membranes, fibers, chips, dishes, and beads. The solid support may be treated, coated or derivatized to facilitate the immobilization of the allele-specific oligonucleotide or target nucleic acid.

In a preferred embodiment, ErbB4 genotyping oligonucleotides may also be immobilized on or synthesized on a solid surface such as a microchip, bead or glass slide (see WO 98/20020 and WO 98/20019). Such immobilized genotyping oligonucleotides may be used in a variety of polymorphism detection assays, including but not limited to probe hybridization and polymerase extension assays. Immobilized ErbB4 genotyping oligonucleotides may comprise an ordered array of oligonucleotides designed to rapidly screen a DNA sample for polymorphisms in multiple genes at the same time.

The genotype or haplotype for the ErbB4 gene of an individual may also be determined by hybridization of a nucleic sample containing one or both copies of the gene to nucleic acid arrays and subarrays such as described in WO 95/11995. The arrays would contain a battery of allele-specific oligonucleotides representing each of the polymorphic sites to be included in the genotype or haplotype.

The identity of polymorphisms may also be determined using a mismatch detection technique, including but not limited to the RNase protection method using riboprobes (e.g. Winter et al., Proc Natl Acad Sci USA 82:7575, 1985; Meyers et al., Science 230:1242, 1985) and proteins which recognize nucleotide mismatches, such as the E. coli mutS protein (e.g. Modrich P. Ann Rev Genet. 25:229-253, 1991). Alternatively, variant alleles can be identified by single strand conformation polymorphism analysis (e.g. Orita et al., Genomics 5:874-879, 1989; Humphries et al., in Molecular Diagnosis of Genetic Diseases, R. Elles, ed., pp. 321-340, 1996) or denaturing gradient gel electrophoresis (DGGE) (e.g. Wartell et at., Nucl Acids Res 18:2699-2706, 1990; Sheffield et al., Proc Natl Acad Sci USA 86:232-236, 1989).

A polymerase-mediated primer extension method may also be used to identify the polymorphism(s). Several such methods have been described, including allele-specific PCR, the “Genetic Bit Analysis” method (e.g. WO 92/15712), the ligase/polymerase mediated genetic bit analysis (e.g. U.S. Pat. No. 5,679,524) and related methods as disclosed in U.S. Pat. Nos. 5,302,509 and 5,945,283. Extended primers containing a polymorphism may be detected by mass spectrometry as described in U.S. Pat. No. 5,605,798. Another primer extension method uses genotyping oligonucleotides hybridizing to a target region located one to several nucleotides downstream of the polymorphic sites.

Multiple polymorphic sites may be investigated by simultaneously amplifying multiple regions of the nucleic acid using sets of allele-specific primers as described in Wallace et al. (WO 89/10414). Two or more sets of allele-specific primer pairs can be used for simultaneous targeting and amplification of two or more regions containing a polymorphic site. Additionally, multiple polymorphisms can be simultaneously detected using two or more differently labeled genotyping oligonucleotides to simultaneously probe the identity of nucleotides at two or more polymorphic sites.

In a preferred embodiment, multiple polymorphisms can be detected by ligase detection reaction. The ligase detection reaction is suitable for detecting multiple SNPs simultaneously as several primer sets can ligate along a gene of interest without the interference encountered in polymerase-based systems (review in Favis et al. 2004, Applications of the Universal DNA Microarray in Molecular Medicine, in Methods in Molecular Medicine Microarrays in Clinical Diagnostics, T. O. Joos and P. Fortina, Editors. 2004, The Humana Press Inc. USA). The regions of interest are amplified then each SNP is simultaneously detected using a thermostable ligase that joins pairs of adjacent oligonucleotides complementary to the sequences of interest. As ligation occurs only when the sequence at the junction between the paired oligonucleotides is exactly complementary to the template sequence, LDR differentiates between wild-type and frameshift or point mutation sequences. The ligation products may be detected using capillary electrophoresis, the discriminating oligonucleotides containing the query base on the 3′ end were labeled with fluorescent dyes. Since non-genomic sequence is added to the LDR oligonucleotides to generate products of specific sizes, the ligation products may be distinguished based on differential label and migration in a capillary-based detection system.

Another aspect provides the compositions comprise oligonucleotide probes and primers designed to specifically hybridize to one or more target regions containing, or that are adjacent to, a polymorphic site. The methods and compositions for establishing the genotype or haplotype of an individual at the novel polymorphic sites described herein are useful for studying the effect of the polymorphisms in the etiology of diseases affected by the expression and function of the ErbB4 protein or lack thereof, studying the efficacy of drugs targeting ErbB4, predicting individual susceptibility to diseases affected by the expression and function of the ErbB4 protein and predicting individual responsiveness to drugs targeting ErbB4.

In another aspect, SNP probes or oligonucleotides, which are useful in classifying people according to their types of genetic variation, are provided. The SNP probes or oligonucleotides can discriminate between alleles of a SNP nucleic acid in conventional allelic discrimination assays. As used herein, the term SNP nucleic acid comprises a nucleotide that is variable within an otherwise identical nucleotide sequence between individuals or groups of individuals, thus, existing as alleles. Such SNP nucleic acids are preferably from about 15 to about 500 nucleotides in length. The SNP nucleic acids may be part of a chromosome, or they may be an exact copy of a part of a chromosome, e.g., by amplification of such a part of a chromosome through PCR or through cloning.

The SNP probes or oligonucleotides are complementary to one allele of the SNP nucleic acid, but not to any other allele of the SNP nucleic acid. The SNP oligonucleotides can discriminate between alleles of the SNP nucleic acid in various ways. For example, under stringent hybridization conditions, an oligonucleotide of appropriate length will hybridize to one allele of the SNP nucleic acid, but not to any other allele of the SNP nucleic acid. The oligonucleotide may be labeled by a radiolabel or a fluorescent label. Alternatively, an oligonucleotide of appropriate length can be used as a primer for PCR, wherein the 3′ terminal nucleotide is complementary to one allele of the SNP nucleic acid, but not to any other allele.

Thus, one embodiment provides an isolated polynucleotide comprising a nucleotide sequence that is a polymorphic variant of a reference sequence for the ErbB4 gene or a fragment thereof. The reference sequence comprises SEQ ID NO: 1 and the polymorphic variant comprise at least one polymorphism, including but not limited to nucleotide: 201 G>A. A particularly preferred polymorphic variant is a naturally occurring sequence of the ErbB4 gene. The isolated polynucleotide, genomic or cDNA fragments described herein comprise at least one novel polymorphic site identified herein and have a length of at least 10 nucleotides and may range up to the full length of the gene.

In describing the polymorphic sites identified herein reference is made to the sense strand of the gene for convenience. However, as recognized by the skilled artisan, nucleic acid molecules containing the ErbB4 gene may be complementary double stranded molecules and thus reference to a particular site on the sense strand refers as well to the corresponding site on the complementary antisense strand. Thus, reference may be made to the same polymorphic site on either strand and an oligonucleotide may be designed to hybridize specifically to either strand at a target region containing the polymorphic site. Thus, the invention also includes single-stranded polynucleotides that are complementary to the sense strand of the ErbB4 genomic variants described herein.

In one embodiment, a kit comprising at least two genotyping oligonucleotides packaged in separate containers is provided. The kit may also contain other components such as hybridization buffer (where the oligonucleotides are to be used as a probe) packaged in a separate container. Alternatively, where the oligonucleotides are to be used to amplify a target region, the kit may contain, packaged in separate containers, a polymerase and a reaction buffer optimized for primer extension mediated by the polymerase, such as PCR.

### Frequency, Association, and Population

In one aspect, a method is provided for determining the frequency of an ErbB4 genotype or haplotype in a population. The method comprises determining the genotype or the haplotype pair for the ErbB4 gene that is present in each member of the population, wherein the genotype or haplotype comprises the nucleotide pair or nucleotide detected at one or more of the polymorphic sites in the ErbB4 gene, including but not limited to the polymorphism at 201 of SEQ ID NO: 1; and calculating the frequency any particular genotype or haplotype is found in the population. The population may be a reference population (e.g. a group of individuals who are predicted to be representative of one or more characteristics of the population group), a family population, a same sex population, a population group (e.g. a group of individuals sharing a common characteristic such as ethnogeographic origin, medical condition, or response to treatment), and a trait population (e.g., a group of individuals exhibiting a trait of interest such as a medical condition or response to a therapeutic treatment).

In another aspect, frequency data for the ErbB4 genotypes and/or haplotypes found in a reference population are used in a method for identifying an association between a trait and an ErbB4 genotype or haplotype. In a preferred embodiment of the method, the trait of interest is a clinical response exhibited by a patient to some therapeutic treatment, for example, response to a drug targeting ErbB4 or response to a therapeutic treatment for a medical condition. As used herein, the term medical condition includes but is not limited to any condition or disease manifested as one or more physical and/or psychological symptoms for which treatment is desirable, and includes previously and newly identified diseases and other disorders.

The association method involves obtaining data on the frequency of the genotype(s) or haplotype(s) of interest in a reference population as well as in a population exhibiting the trait. Frequency data for one or both of the reference and trait populations may be obtained by genotyping or haplotyping each individual in the populations using one of the methods described above. The haplotypes for the trait population may be determined directly or, alternatively, by the predictive genotype to haplotype approach described above.

The frequency data for the reference and/or trait populations can be obtained by accessing previously determined frequency data, which may be in written or electronic form. For example, the frequency data may be present in a database that is accessible by a computer. Once the frequency data is obtained the frequencies of the genotype(s) or haplotype(s) of interest in the reference and trait populations are compared. In a preferred embodiment, the frequencies of all genotypes observed in the reference populations are compared. If a particular genotype or haplotype for the ErbB4 gene is more frequent in the trait population than in the reference population at a statistically significant amount, then the trait is predicted to be associated with that ErbB4 genotype or haplotype.

In another aspect, a detectable genotype or haplotype that is in linkage disequilibrium with the ErbB4 genotype or haplotype of interest may be used as a surrogate marker. A genotype that is in linkage disequilibrium with a ErbB4 genotype may be discovered by determining if a particular genotype or haplotype for the ErbB4 gene is more frequent in the population that also demonstrates the potential surrogate marker genotype than in the reference population at a statistically significant amount, then the marker genotype is predicted to be associated with that ErbB4 genotype or haplotype and then can be used as a surrogate marker in place of the ErbB4 genotype.

In order to deduce a correlation between clinical response to a treatment and an ErbB4 genotype or haplotype, it is necessary to obtain data on the clinical responses exhibited by a population of individuals who received the treatment, hereinafter the “clinical population”. This clinical data may be obtained by analyzing the results of a clinical trial that has already been run and/or the clinical data may be obtained by designing and carrying out one or more new clinical trials. As used herein, the term clinical trial means any research study designed to collect clinical data on responses to a particular treatment, and includes but is not limited to phase I, phase II and phase III clinical trials. Standard methods are used to define the patient population and to enroll subjects. As used herein, the term clinical response means any or all of the following: a quantitative measure of the response, no response, and adverse response (i.e., side effects).

It is preferred that the individuals included in the clinical population have been graded for the existence of the medical condition of interest. This is important in cases where the symptom(s) being presented by the patients can be caused by more than one underlying condition, and where treatment of the underlying conditions are not the same. An example of this would be where patients experience breathing difficulties that are due to either asthma or respiratory infections. If both sets were treated with an asthma medication, there would be a spurious group of apparent non-responders that did not actually have asthma. These people would affect the ability to detect any correlation between genotype/haplotype and treatment outcome. This grading of potential patients could employ a standard physical exam or one or more lab tests. Alternatively, grading of patients could use haplotyping for situations where there is a strong correlation between haplotype pair and disease susceptibility or severity.

The therapeutic treatment of interest is administered to each individual in the trial population and each individual's response to the treatment is measured using one or more predetermined criteria. It is contemplated that in many cases, the trial population will exhibit a range of responses and that the investigator will choose the number of responder groups (e.g., low, medium, high) made up by the various responses.

The ErbB4 gene for each individual in the clinical or trial population is genotyped and/or haplotyped, which may be done before or after administering the treatment. After both the clinical and polymorphism data have been obtained, correlations between individual response and ErbB4 genotype or haplotype content are created. Correlations may be produced in several ways. In one method, individuals are grouped by their ErbB4 genotype or haplotype (or haplotype pair) (also referred to as a polymorphism group), and then the averages and standard deviations of clinical responses exhibited by the members of each polymorphism group are calculated.

These results are then analyzed to determine if any observed variation in clinical response between polymorphism groups is statistically significant. Statistical analysis methods which may be used are described in L. D. Fisher and G. vanBelle, “Biostatistics: A Methodology for the Health Sciences”, Wiley-Interscience (New York) 1993. This analysis may also include a regression calculation of which polymorphic sites in the ErbB4 gene give the most significant contribution to the differences in phenotype.

Correlations may also be analyzed using predictive models based on error-minimizing optimization algorithms. One of many possible optimization algorithms is a genetic algorithm (e.g. R. Judson, “Genetic Algorithms and Their Uses in Chemistry” in Reviews in Computational Chemistry, Vol. 10, pp. 1-73, K. B. Lipkowitz and D. B. Boyd, eds. (VCH Publishers, New York, 1997). Simulated annealing (e.g. Press et al., “Numerical Recipes in C: The Art of Scientific Computing”, Cambridge University Press (Cambridge) 1992, Ch. 10), neural networks (e.g. E. Rich and K. Knight, “Artificial Intelligence”, 2nd Edition (McGraw-Hill, New York, 1991, Ch. 18), standard gradient descent methods (e.g. Press et al., supra Ch. 10), or other global or local optimization approaches (see discussion in Judson, supra) could also be used. In addition, the correlation can be found using a genetic algorithm approach as described in PCT Application entitled “Methods for Obtaining and Using Haplotype Data”, filed Jun. 26, 2000.

Statistical analysis can be performed by the use of a general linear model with a Bonferoni correction and/or a bootstrapping or permutation method that simulates the genotype-phenotype correlation many times and calculates a significance value. When many polymorphisms are being analyzed a correction to factor may be performed to correct for a significant association that might be found by chance. For statistical methods for use in the methods described herein, see: Statistical Methods in Biology, 3^{rd} edition, Bailey N T J, Cambridge Univ. Press (1997); Introduction to Computational Biology, Waterman M S, CRC Press (2000) and Bioinformatics, Baxevanis A D and Ouellette B F F editors (2001) John Wiley & Sons, Inc.

From the analyses described above, a mathematical model may be readily constructed by the skilled artisan that predicts clinical response as a function of ErbB4 genotype or haplotype content. Preferably, the model is validated in one or more follow-up clinical trials designed to test the model.

The identification of an association between a clinical response and a genotype, haplotype or haplotype pair for the ErbB4 gene may be the basis for designing a diagnostic method to determine those individuals who will or will not respond to the treatment, or alternatively, will respond at a lower level and thus may require more treatment, i.e., a greater dose of a drug or different drug. The diagnostic method may take one of several forms: for example, a direct DNA test (i.e., genotyping or haplotyping one or more of the polymorphic sites in the ErbB4 gene), a serological test, or a physical exam measurement. The only requirement is that there be a good correlation between the diagnostic test results and the underlying ErbB4 genotype or haplotype that is in turn correlated with the clinical response. In a preferred embodiment, this diagnostic method uses the predictive genotyping method described above.

A computer may implement any or all analytical and mathematical operations involved in practicing the methods of the present invention. In addition, the computer may execute a program that generates views (or screens) displayed on a display device and with which the user can interact to view and analyze large amounts of information relating to the ErbB4 gene and its genomic variation, including chromosome location, gene structure, and gene family, gene expression data, polymorphism data, genetic sequence data, and clinical data population data (e.g., data on ethnogeographic origin, clinical responses, genotypes, and haplotypes for one or more populations). The ErbB4 polymorphism data described herein may be stored as part of a relational database (e.g., an instance of an Oracle database or a set of ASCII flat files). These polymorphism data may be stored on the computer's hard drive or may, for example, be stored on a CD-ROM or on one or more other storage devices accessible by the computer. For example, the data may be stored on one or more databases in communication with the computer via a network.

In another embodiment, a method is provided for identifying an association between a genotype or haplotype and a trait. In preferred embodiments, the trait is susceptibility to a disease, severity of a disease, the staging of a disease or response to a drug. Such methods have applicability in developing diagnostic tests and therapeutic treatments for all pharmacogenetic applications where there is the potential for an association between a genotype and a treatment outcome including efficacy measurements, pharmacokinetic measurements and side effect measurements.

### GLOSSARY AND DEFINITIONS

The following glossary and definitions are provided to facilitate understanding of certain terms used frequently in this specification.

As used herein the term psychotic disorder shall mean any pathologic psychological condition in which psychotic symptoms can or do occur and includes, but is not limited to the following; (also see, Diagnostic and Statistical Manual of Mental Disorders 4^{th} Edition (DSM-IV) Francis A editor, American Psychiatric Press, Wash, D.C., 1994)

### Schizophrenic Disorders

### Schizophrenia, Catatonic, Subchronic, (295.21),

### Schizophrenia, Catatonic, Chronic (295.22),

Schizophrenia, Catatonic, Subchronic with Acute Exacerbation (295.23),
Schizophrenia, Catatonic, Chronic with Acute Exacerbation (295.24),

### Schizophrenia, Catatonic, in Remission (295.55),

### Schizophrenia, Catatonic, Unspecified (295.20),

### Schizophrenia, Disorganized, Subchronic (295.11),

### Schizophrenia, Disorganized, Chronic (295.12),

Schizophrenia, Disorganized, Subchronic with Acute Exacerbation (295.13),
Schizophrenia, Disorganized, Chronic with Acute Exacerbation (295.14),

### Schizophrenia, Disorganized, in Remission (295.15),

### Schizophrenia, Disorganized, Unspecified (295.10),

### Schizophrenia, Paranoid, Subchronic (295.31),

### Schizophrenia, Paranoid, Chronic (295.32),

Schizophrenia, Paranoid, Subchronic with Acute Exacerbation (295.33),
Schizophrenia, Paranoid, Chronic with Acute Exacerbation (295.34),

### Schizophrenia, Paranoid, in Remission (295.35),

### Schizophrenia, Paranoid, Unspecified (295.30),

### Schizophrenia, Undifferentiated, Subchronic (295.91),

### Schizophrenia, Undifferentiated, Chronic (295.92),

Schizophrenia, Undifferentiated, Subchronic with Acute Exacerbation (295.93),
Schizophrenia, Undifferentiated, Chronic with Acute Exacerbation (295.94),

### Schizophrenia, Undifferentiated, in Remission (295.95),

### Schizophrenia, Undifferentiated, Unspecified (295.90),

### Schizophrenia, Residual, Subchronic (295.61),

### Schizophrenia, Residual, Chronic (295.62),

Schizophrenia, Residual, Subchronic with Acute Exacerbation (295.63),
Schizophrenia, Residual, Chronic with Acute Exacerbation (295.94),

### Schizophrenia, Residual, in Remission (295.65),

### Schizophrenia, Residual, Unspecified (295.60),

### Delusional (Paranoid) Disorder (297.10),

### Brief Reactive Psychosis (298.80),

### Schizophreniform Disorder (295.40),

### Schizoaffective Disorder (295.70),

### Induced Psychotic Disorder (297.30),

### Psychotic Disorder NOS (Atypical Psychosis) (298.90)

### Affective Disorders

Major Depressive Disorder, Severe with Psychotic Features (296.33)
Bipolar I Disorder, Single Manic Episode, Severe with Psychotic Features (296.23)

### Bipolar I Disorder, Most Recent Episode Hypomanic (296.43)

Bipolar I Disorder, Most Recent Episode Manic, Severe with Psychotic Features (296.43)
Bipolar I Disorder, Most Recent Episode Mixed, Severe with Psychotic Features (296.63)
Bipolar I Disorder Most Recent Episode Depressed, Severe with Psychotic Features (296.53)

### Bipolar I Disorder, Most Recent Episode Unspecified (296.89)

### Bipolar II Disorder (296.89)

### Cyclothymic Disorder (301.13)

### Bipolar Disorder NOS (366)

### Mood Disorder Due To (General Medical Condition) (293.83)

### Mood Disorder NOS (296.90)

### Conduct Disorder, Solitary Aggressive Type (312.00),

### Conduct Disorder, Undifferentiated Type (312.90),

### Tourette's Disorder (307.23),

### Chronic Motor Or Vocal Tic Disorder (307.22),

### Transient Tic Disorder (307.21),

### Tic Disorder NOS (307.20),

### Psychoactive Substance Use Disorders

### Alcohol Withdrawal Delirium (291.00),

### Alcohol Hallucinosis (291.30),

Alcohol Dementia Associated with Alcoholism (291.20),

### Amphetamine or Similarly Acting Sympathomimetic Intoxication (305.70),

### Amphetamine or Similarly Acting Sympathomimetic Delirium (292.81),

### Amphetamine or Similarly Acting Sympathomimetic Delusional Disorder (292.11),

### Cannabis Delusional Disorder (292.11),

### Cocaine Intoxication (305.60),

### Cocaine Delirium (292.81),

### Cocaine Delusional Disorder (292.11),

### Hallucinogen Hallucinosis (305.30),

### Hallucinogen Delusional Disorder (292.11),

### Hallucinogen Mood Disorder (292.84),

Hallucinogen Post hallucinogen Perception Disorder (292.89),

### Phencyclidine (PCP) or Similarly Acting Arylcyclohexylamine Intoxication (305.90),

### Phencyclidine (PCP) or Similarly Acting Arylcyclohexylamine Delirium (292.81),

### Phencyclidine (PCP) or Similarly Acting Arylcyclohexylamine Delusional Disorder (292.11),

### Phencyclidine (PCP) or Similarly Acting Arylcyclohexylamine Mood Disorder (292.84),

### Phencyclidine (PCP) or Similarly Acting Arylcyclohexylamine Organic Mental Disorder NOS (292.90),

### Other or Unspecified Psychoactive Substance Intoxication (305.90),

### Other or Unspecified Psychoactive Substance Delirium (292.81),

### Other or Unspecified Psychoactive Substance Dementia (292.82),

### Other or Unspecified Psychoactive Substance Delusional Disorder (292.11),

### Other or Unspecified Psychoactive Substance Hallucinosis (292.12),

### Other or Unspecified Psychoactive Substance Mood Disorder (292.84),

### Other or Unspecified Psychoactive Substance Anxiety Disorder (292.89),

### Other or Unspecified Psychoactive Substance Personality Disorder (292.89),

### Other or Unspecified Psychoactive Substance Organic Mental Disorder NOS (292.90)

### Delirium (293.00),

### Dementia (294.10),

### Obsessive Compulsive Disorder (300.30),

### Intermittent Explosive Disorder (312.34),

### Impulse Control Disorder NOS (312.39)

### Personality Disorders

### Personality Disorder, Paranoid (301.00),

### Personality Disorder, Schizoid (301.20),

### Personality Disorder, Schizotypal (301.22),

### Personality Disorder, Antisocial (301.70),

### Personality Disorder, Borderline (301.83)

The term antipsychotic medication or drug as used herein means any medication used to decrease or ameliorate the symptoms of psychosis in a person with a psychotic disorder and includes, but is not limited to the following compounds: Acetophenazine Maleate; Alentemol Hydrobromide; Alpertine; Azaperone; Batelapine Maleate; Benperidol; Benzindopyrine Hydrochloride; Brofoxine; Bromperidol; Bromperidol Decanoate; Butaclamol Hydrochloride; Butaperazine; Butaperazine Maleate; Carphenazine Maleate; Carvotroline Hydrochloride; Chlorpromazine; Chlorpromazine Hydrochloride; Chlorprothixene; Cinperene; Cintriamide; Clomacran Phosphate; Clopenthixol; Clopimozide; Clopipazan Mesylate; Cloroperone Hydrochloride; Clothiapine; Clothixamide Maleate; Clozapine; Cyclophenazine Hydrochloride; Droperidol; Etazolate Hydrochloride; Fenimide; Flucindole; Flumezapine; Fluphenazine Decanoate; Fluphenazine Enanthate; Fluphenazine Hydrochloride; Fluspiperone; Fluspirilene; Flutroline; Gevotroline Hydrochloride; Halopemide; Haloperidol; Haloperidol Decanoate; Iloperidone; Imidoline Hydrochloride; Lenperone; Mazapertine Succinate; Mesoridazine; Mesoridazine Besylate; Metiapine; Milenperone; Milipertine; Molindone Hydrochloride; Naranol Hydrochloride; Neflumozide Hydrochloride; Ocaperidone; Olanzapine; Oxiperomide; Penfluridol; Pentiapine Maleate; Perphenazine; Pimozide; Pinoxepin Hydrochloride; Pipamperone; Piperacetazine; Pipotiazine Palmitate; Piquindone Hydrochloride; Prochlorperazine Edisylate; Prochlorperazine Maleate; Promazine Hydrochloride; Quetiapine; Remoxipride; Remoxipride Hydrochloride; Risperidone; Rimcazole Hydrochloride; Seperidol Hydrochloride; Sertindole; Setoperone; Spiperone; Thioridazine; Thioridazine Hydrochloride; Thiothixene; Thiothixene Hydrochloride; Tioperidone Hydrochloride; Tiospirone Hydrochloride; Trifluoperazine Hydrochloride; Trifluperidol; Triflupromazine; Triflupromazine Hydrochloride; and Ziprasidone Hydrochloride.

Allele—A particular form of a genetic locus, distinguished from other forms by its particular nucleotide sequence.

Gene—A segment of DNA that contains all the information for the regulated biosynthesis of an RNA product, including promoters, exons, introns, and other untranslated regions that control expression.

Genotype—An unphased 5′ to 3′ sequence of nucleotide pair(s) found at one or more polymorphic sites in a locus on a pair of homologous chromosomes in an individual.

Genotyping—A process for determining a genotype of an individual.

Haplotype—A 5′ to 3′ sequence of nucleotides found at one or more polymorphic sites in a locus on a single chromosome from a single individual.

Haplotype pair—The two haplotypes found for a locus in a single individual.

Haplotyping—A process for determining one or more haplotypes in an individual and includes use of family pedigrees, molecular techniques and/or statistical inference.

Identity—A relationship between two or more polypeptide sequences or two or more polynucleotide sequences, determined by comparing the sequences. In general, identity refers to an exact nucleotide to nucleotide or amino acid to amino acid correspondence of the two polynucleotide or two polypeptide sequences, respectively, over the length of the sequences being compared.

Isolated—As applied to a biological molecule such as RNA, DNA, oligonucleotide, or protein, isolated means the molecule is substantially free of other biological molecules such as nucleic acids, proteins, lipids, carbohydrates, or other material such as cellular debris and growth media. Generally, the term “isolated” is not intended to refer to a complete absence of such material or to absence of water, buffers, or salts, unless they are present in amounts that substantially interfere with the methods of the present invention.

Linkage disequilibrium—A situation in which some combinations of genetic markers occur more or less frequently in the population than would be expected from their distance apart. It implies that a group of markers has been inherited coordinately. It can result from reduced recombination in the region or from a founder effect, in which there has been insufficient time to reach equilibrium since one of the markers was introduced into the population.

Locus—A location on a chromosome or DNA molecule corresponding to a gene or a physical or phenotypic feature.

Phased—As applied to a sequence of nucleotide pairs for two or more polymorphic sites in a locus, phased means the combination of nucleotides present at those polymorphic sites on a single copy of the locus is known.

Unphased—As applied to a sequence of nucleotide pairs for two or more polymorphic sites in a locus, unphased means the combination of nucleotides present at those polymorphic sites on a single copy of the locus is not known.

Polynucleotide—Any polyribonucleotide (RNA) or polydeoxyribonucleotide (DNA), which may be unmodified or modified RNA or DNA. Polynucleotides include, without limitation, single- and double-stranded DNA, DNA that is a mixture of single- and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions. In addition, polynucleotide refers to triple-stranded regions comprising RNA or DNA or both RNA and DNA. The term polynucleotide also includes DNAs or RNAs containing one or more modified bases and DNAs or RNAs with backbones modified for stability or for other reasons. Modified bases include, for example, tritylated bases and unusual bases such as inosine. A variety of modifications may be made to DNA and RNA; thus, polynucleotide embraces chemically, enzymatically or metabolically modified forms of polynucleotides as typically found in nature, as well as the chemical forms of DNA and RNA characteristic of viruses and cells. “Polynucleotide” also embraces relatively short polynucleotides, often referred to as oligonucleotides.

Polypeptide—Any polypeptide comprising two or more amino acids joined to each other by peptide bonds or modified peptide bonds, i.e. peptide isosteres. Polypeptide refers to both short chains, commonly referred to as peptides, oligopeptides or oligomers, and to longer chains, generally referred to as proteins. Polypeptides may contain amino acids other than the 20 gene-encoded amino acids. Polypeptides include amino acid sequences modified either by natural processes, such as post-translational processing, or by chemical modification techniques that are well known in the art. Such modifications are well described in basic texts and in more detailed monographs, as well as in a voluminous research literature. Modifications may occur anywhere in a polypeptide, including the peptide backbone, the amino acid side-chains and the amino or carboxyl termini.

### EXAMPLE

### Example 1

### Patients, Cohorts, and Phenotypes

The paliperidone treated arms of clinical trial Nos. 1-4, were pooled and examined for genetic association. The trial Nos. 1-3 were paliperidone ER phase III studies and trial No. 4 was a paliperidone palmitate phase II/III study. To confirm results of the initial genetic association, additional paliperidone treated cohorts, including paliperidone palmitate phase III clinical trial Nos 5-8 and paliperidone ER phase III/IV trial Nos. 9 and 10 were evaluated. The results were further examined with the placebo arms of trial Nos. 1-4 and 6-10, the olanzapine arms of trial Nos. 1-3 and 11, the quetiapine arm of trial No. 9, and the Risperdal Consta arm of the trials No. 5 and 11.

Trial Nos. 1 to 3 were randomized, double-blind, placebo and active controlled phase III trials that were designed to evaluate efficacy and safety of paliperidone-ER in the acute treatment of subjects with schizophrenia. Schizophrenic patients experiencing active symptoms, with Positive and Negative Symptoms of Schizophrenia (PANSS) score=70-120, were enrolled and randomized to different treatment groups. The patients received paliperidone-ER of 3 mg, 6 mg, 9 mg, 12 mg, or 15 mg, olanzapine of 10 mg or placebo tablet once daily for 6 weeks. Primary efficacy variable/Primary Time point was changed in the total PANSS score from baseline to endpoint. The baseline value was the measurement from the last visit prior to or including the first day of dosing with double-blind study medication, and the endpoint value was the measurement at the end of the double-blind phase or the last post-baseline observation during the double-blind phase. Detailed clinical study design and outcomes of trial Nos. 1-3 were described elsewhere (Marder et al., Biol Psychiatry. 2007, 62:1363-70; Davidson et al., Schizophr Res. 2007, 93:117-30; Kane et al., Schizophr Res. 2007, 90:147-61. Epub).

Trial No. 4 was a randomized, double-blind, placebo-controlled phase II/III study to evaluate the efficacy and safety of long-acting injections of paliperidone palmitate at fixed doses of 50 and 100 mg equivalent in subjects with schizophrenia. Subjects were required to have a total PANSS score between 70 and 120 at Screening and between 60 and 120 on Day 1 prior to the start of double-blind treatment. The primary efficacy variable was the change in total PANSS score from the start of the double-blinded treatment period to the last post-randomization assessment in the double-blinded treatment period.

Trial No. 5 was a randomized, double blind, parallel-group comparative study of Paliperidone Palmitate and Risperdal Consta. Subjects with a PANSS total score of 60-120 were enrolled. and received flexible doses of Paliperidone Palmitate of 25, 50, 75, or 100 mg eq. every 4 weeks or flexible doses of Risperdal Consta of 25, 37.5, or 50 mg every 2 weeks. Subjects received 1-6 mg/day oral Risperidone supplement in the Risperdal Consta arm or placebo in the Paliperidone Palmitate arm during the first 4 weeks of the study. This was long-term 53 week study during which efficacy, safety and PK were assessed periodically. The primary efficacy variable was the change from baseline to end point in total PANSS score. The PANSS score at 13 weeks were used for genetic study.

Trials No. 6 and 7 were a 13 week, randomized, double-blinded, placebo-controlled, parallel-group, dose-response study to evaluate efficacy and safety of fixed doses of Paliperidone Palmitate of 25, 50, 100, or 150 mg eq. in subjects with schizophrenia. Subjects with the PANSS total score of 70-120 were enrolled. The primary efficacy variable was the change in total PANSS score from baseline to last post-randomization assessment in the double-blinded treatment period.

Trial No. 8 was a 13-week, double-blind, randomized, placebo-controlled, parallel-group, multicenter, dose-response study to compare efficacy, safety and tolerability of 3 fixed doses of Paliperidone Palmitate (25, 100, or 150 mg eq.) with those of placebo. At the beginning of the double-blind treatment period, subjects with schizophrenia were randomly assigned in equal numbers to 1 of 4 treatment groups of Paliperidone Palmitate 25, 100, or 150 mg eq. or placebo. All subjects were to receive an injection in the deltoid muscle of Paliperidone Palmitate 150 mg eq. or the corresponding placebo on Day 1. The primary efficacy variable/time point was the change in the PANSS total score from baseline to end point.

Trial No. 9 was a 6-week double-blind, placebo-controlled study that compared the effects of Paliperidone-ER and Quetiapine monotherapy followed by a period of optional polypharmacy use in patients with a recent acute exacerbation of schizophrenia requiring hospitalization. Subjects were randomized to Paliperidone-ER, Quetiapine or placebo in a 2:2:1 ratio. The study consisted of a 2-week monotherapy phase followed by a 4-week additive-therapy phase. Target doses were 9 or 12 mg/day of Paliperidone-ER and 600 or 800 mg/day of Quetiapine. The change in PANSS total score at the end of study was used for genetic study.

Trial No. 10 was a randomized, double-blind, placebo-controlled, parallel group, flexible dose study with 2 treatment groups: placebo and Paliperidone-ER (3 to 12 mg/day with 3 mg dose in- or decrements), with a ratio of 1:2. The study consisted of a screening and washout period of 5 days, and a 6-week double-blind phase. Following the double-blind phase, eligible subjects could enter the 52-week open-label extension with Paliperidone-ER. The major efficacy variables/Time points were the changes in the PANSS total score; Personal and Social Performance Scale (PSP); Clinical Global Impression-Severity (CGI-S); and Schizophrenia Quality of Life Scale Revision 4 (SQLS-R4) score. All changes are from baseline to endpoint.

Trial No. 11 was an open-label, randomized, international, multicenter, flexible-dose study conducted in schizophrenic or schizoaffective subjects with a total duration of 12 months. The study was divided into 2 parts: the first part included Week 1 to 13 and the second part included Week 14 to Week 53. Subjects were titrated to their optimal oral dose during run-in and converted to a pre-defined dose of Risperidone long-acting injections of 25, 50 or 75 mg every 2 weeks, or continued on their last run-in dose of Olanzapine tablets of 5, 10, 15 or 20 mg once daily.

DNA collected from subjects who consented to the optional pharmacogenomic component of the above clinical trials were analyzed in the genetic study. Briefly, blood samples were collected from subjects and extracted for genomic DNA. About 2 μg of genomic DNA from each subject was genotyped by a custom SNP microarray based on Infinium II platform (Illumina, San Diego, Calif.). The custom microarray contains 29,080 oligonucleotides, including oligonucleotides corresponding to rs6435681 in the ErbB4 gene. The average genotyping call rate for the successful loci was 99.73% and the reproducible rate was 99.6% based on genotyping data of 67 pairs of duplicated samples. Mendelian consistency was 2.6×10⁻⁵ based on genotyping data of 2 trios.

To confirm the results of the initial genetic association studies, a multiplex ligase detection reaction assay was used for genotyping. About 150 ng of genomic DNA was used for ligase-based genotyping assay according to Luo et al., 1996 and Favis et al. 2000 (Favis et al., 2000, Nat. Biotechnol. 18: 561-4; Luo et al., 1996, Nucleic Acids Res. 24:3071-8). The clinical endpoints for pharmacogenomic analysis from baseline to the end of the double blind treatment period (for trials 1-4, 6-10) or to the end of the first 13 weeks in trial Nos. 5 and 11 were the changes in the following 7 scales: PANSS total scores (Kay S R et al. 1987 Schizophrenia Bulletin 13; 2:261-276), 5 PANSS Marder factor scores (Marder et al., 1997, J Clin Psychiatry 58: 538-46), and Lindenmayer excitement subscale (Lindenmayer et al. 2004, Schizophr Res. 68:331-7).

### Example 2

### Statistical Analysis

The initial genetic association study was conducted with 685 Paliperidone-treated subjects, including 543 Caucasians, from trial Nos. 1-4. All analyses were conducted using the SAS software package (version 9.1.3). Seven efficacy endpoints were considered as described in example 1. General linear model was applied for examining covariates and the phenotype-genotype associations. A number of variables were examined, including gender, race, country, trial, dosage, age at diagnosis, duration of disease, disease subtype, and score at baseline. Significant covariates which were age at diagnosis, country, dosage, and score at baseline, were included in the final model. Covariates of age and score at baseline were included as continuous variables, and covariates of country and dose were included as categorical variables. Also, 4 genetic models of general, additive, dominant, and recessive models were considered. For each efficacy endpoint and each genetic model, the association was tested for rs6435681 or SNP at the polymorphism of 201 G>A in SEQ ID NO:1. False discovery rate was calculated based on the empirical distribution of the p-values from association tests. The analyses were conducted in Caucasian subjects only. Additional analyses were further examined in subjects of all races.

### Example 3

### Efficacy Results

The initial genetic association study, including subjects from trials 1-4, identified the change in PANSS total score from baseline to end of study was significantly associated with SNP marker rs6435681 in the ErbB4 gene (unadjusted p-value: 8.2×10⁻⁷; Bonferroni adjusted p-value: 0.02). Patients with AA genotype at this SNP had less reduction in PANSS total score from baseline, prior to double-blind treatment, as compared to patients with GG/GA genotypes. The mean reduction in PANSS total score was 17.8 in patients with GG or GA genotype (N=659) and 0.6 in patients with AA genotype (N=25). Twenty-nine percent of the patients with GG/GA genotype displayed a 30% or higher reduction in PANSS total score from baseline, and 8% of the patients with AA genotype displayed similar extent of reduction in PANSS total score.

In the placebo arm of these trials, patients with AA genotype at rs6435681 had greater reduction in PANSS total score as compared to patient with GG/GA genotypes (p-value: 0.03). The mean reduction in PANSS total score was 13.7 in patients with AA genotype (N=16) and 0.6 in patients with GG or GA genotype (N=242) in placebo arm. Nineteen percent of the patients with AA genotype displayed a 30% or higher reduction in PANSS total score and 13% of the patients with GG or GA genotype displayed similar extent of reduction in placebo arm.

To confirm the results of the initial genetic studies, further analysis were conducted with the same clinical endpoints (i.e. PANSS Total, Marder positive factor, and Marder disorganized thoughts factor) and the same genetic model (i.e. recessive) from the initial genetic association study. The analyses were performed in subjects of all races. Similar genetic effect was observed in trial No. 8 with an unadjusted P value for the change of total PANSS score of 0.026 in treated patients and 0.017 in placebo. Although the tests were not statistically significant after adjusted for multiple testing, the same trend of the change of total PANSS across the genotypic groups as the original study was observed in trial No. 8. In addition, an exploratory analysis on change of Personal and Social Performance (PSP) scale showed strong evidence of association with the SNP at the same polymorphic site at 201 of SEQ ID NO: 1 (P=0.008) in treated patients.

Similar genetic effect was not observed in pooled replication studies of trial Nos. 5-7 and 9-10 (p value is not significant). This may due to less optimal dosing regimen or relatively small sample size. In addition, similar genetic effect was not observed in subjects treated with Olanzapine, Risperidone, and Quetiapine. This may be due to limited sample size. These results of Example 3 indicate that the SNP at the polymorphic site of 201 in SEQ ID NO: 1 may be used to differentiate efficacy of Paliperidone from other antipsychotic agents.

### Example 4

### Additional Phenotype Analysis

To examine whether the polymorphism at 201 G>A in SEQ ID NO: 1 was due to phenotypes other than the genetic efficacy endpoint that were correlated with the genotype at this SNP, the distributions of possibly related phenotypes were further examined. These phenotypes were grouped into 4 categories.
1. Demographics and psychiatric history: gender, age, BMI, race, country, age of diagnosis, disease duration, disease subtype, use of anti-depressant, use of anti-histamine, number of prior hospitalization, baseline PANSS total score, and baseline Clinical Global Impression-Severity (CGI-S) scale.
2. Drug exposure: trial, treatment arm, withdraw/early termination.
3. Adverse events: treatment emergent AE, severe AE, AE leading to discontinuation, and extrapyramidal symptom (EPS) related AE.
4. Pharmacokinetics: AUC (area under the curve of paliperidone in plasma) derived from raw and dose-adjusted concentration.

The distribution of each phenotype in the subjects with AA genotype was compared with its distribution in the subjects with non-AA genotype, i.e. GG or GA. When the phenotype distribution was categorical, a Chi-square test was conducted to compare the phenotype distributions between AA and non-AA genotypes unless the categorical phenotype had too many levels and the degree of freedom was too high for a Chi-square test. When the phenotype distribution was continuous, either an ANOVA or a Wilcoxon rank-sum test was conducted to compare the distributions between AA and non-AA genotypes. In addition, an ANOVA test was conducted when the phenotype distribution was approximately normal and a Wilcoxon rank-sum tests was conducted when the normal assumption was grossly violated. The analysis was performed in the Paliperidone and placebo arms separately and in Caucasians and subjects of all races separately.

The only phenotype that was significantly correlated with the genotype at polymorphism of 201 G>A in SEQ ID NO: 1 was withdraw/early termination. In the Paliperidone arm, 47.8% of the subjects with AA genotype had higher withdrawal/early termination rate compared to 27.4% of the subjects with non-AA genotype (p-value=0.05); and 48% of the Caucasians had higher withdrawal/early termination rate compared to 31.6% of the subjects of all races (p-value=0.12). In the placebo arm, 33.3% of the subjects with AA genotype had lower withdrawal/early termination rate compared with 65.4% of the subjects with non-AA genotype (p-value=0.03); and in 31.3% of the Caucasians had lower withdrawal/early termination rate compared with 62% of the subjects of all races (p-value=0.02). Reasons for the withdrawals/early terminations were further examined to explore possible explanations for this. Majority of the withdrawals/early terminations were due to lack of efficacy, which was the same trend as those of Example 3. No significant correlation was found for other phenotypes. The results of Example 4 indicates that the validity of the polymorphism at 201 G>A in SEQ ID NO: 1 is not affected by the phenotypes examined herein.

What is claimed is:

1. A method for predicting likelihood of a patient responding to treatment with an atypical antipsychotic drug selected from the group consisting of Paliperidone, and pharmaceutically acceptable salts and esters thereof, comprising:
a) analyzing nucleotide sequences of two alleles of the ErbB4 gene in said patient;
b) determining a genotype at a polymorphic site in the ErbB4 gene, wherein said polymorphic site is at 201 in SEQ ID NO: 1; and
c) identifying the patient with the genotype of GA or GG as being more likely to respond to the drug, and the patient with the genotype of AA as being less likely to respond to the drug.

2. The method of claim 1, wherein the atypical antipsychotic drug is selected from the group consisting of Paliperidone and Paliperidone Palmitate.

3. The method of claim 1, wherein said nucleotide sequences are determined by the method selected from the group consisting of hybridization, sequencing, primer extension, ligase detection reaction, and cleavage method.

4. The method of claim 3, wherein said nucleotide sequences are determined by ligase detection reaction.

5. The method of claim 3, wherein said nucleotide sequences are determined by hybridization.

6. A method for predicting a patient being less likely to respond to a treatment with an atypical antipsychotic drug selected from the group consisting of Paliperidone, and pharmaceutically acceptable salts and esters thereof, comprising:
a) analyzing nucleotide sequences of two alleles of the ErbB4 gene in said patient;
b) determining a genotype at a polymorphic site in the ErbB4 gene, wherein said polymorphic site is at 201 in SEQ ID NO: 1; and
c) identifying the patient with the genotype of AA as being less likely to respond to the drug.

7. The method of claim 6, wherein the atypical antipsychotic drug is selected from the group consisting of Paliperidone and Paliperidone Palmitate.

8. A method for predicting a patient being more likely to respond to a treatment with an atypical antipsychotic drug selected from the group consisting of Paliperidone, and pharmaceutically acceptable salts and esters thereof, comprising:
a) analyzing nucleotide sequences of two alleles of the ErbB4 gene in said patient;
b) determining a genotype of a polymorphic site in the ErbB4 gene, wherein said polymorphic site is at 201 in SEQ ID NO: 1; and
c) identifying the patient with the genotype of GA or GG as being more likely to respond to the drug.

9. The method of claim 8, wherein the atypical antipsychotic drug is selected from the group consisting of Paliperidone and Paliperidone Palmitate.

10. A method for predicting whether a patient in need of treatment with antipsychotic drug is likely to display placebo response, comprising:
a) analyzing nucleotide sequences of two alleles of the ErbB4 gene in said patient;
b) determining a genotype at a polymorphic site in the ErbB4 gene, wherein said polymorphic site is at 201 in SEQ ID NO: 1; and
c) identifying the patient with the genotype of AA as being likely to display placebo response.

11. A method for selecting a subject for a clinical study of an antipsychotic medication comprising:
a) analyzing nucleotide sequences of two alleles of the ErbB4 gene in said patient;
b) determining a genotype at a polymorphic site in the ErbB4 gene, wherein the polymorphic site is at 201 in SEQ ID NO: 1; and
c) identifying the subject with the genotype of AA as being likely to display placebo response.

12. The method of claim 9, further comprising excluding the subject with the genotype of AA from the clinical study of an antipsychotic medication.

13. A method of treating a patient in need of a psychotic treatment comprising:
a) obtaining a DNA sample from the patient;
b) analyzing nucleotide sequences of the ErbB4 gene in the patient;
c) determining a genotype of a polymorphic site in the ErbB4 gene, wherein the polymorphic site is at 201 in SEQ ID NO: 1; and
d) treating the patient with the genotype of GA or GG with an atypical antipsychotic drug selected from the group consisting of Paliperidone, and pharmaceutically acceptable salts and esters thereof.

14. The method of claim 13, wherein the atypical antipsychotic drug is selected from the group consisting of Paliperidone and Paliperidone Palmitate.

15. A kit for use in determining treatment strategy for a patient with a psychotic disorder comprising:
a) a polynucleotide able to recognize and bind to portion of the ErbB4 gene;
b) a container suitable for containing said polynucleotide and a DNA sample from the d patient wherein the polynucleotide can contact the ErbB4 gene; and means to detect hybridization of the polynucleotide with the ErbB4 gene.

16. The kit of claim 15, further comprising instructions for use.


SEQUENCE LISTING (1 sequence)

SEQ ID NO: 1 - DNA, 701 bp, Homo sapiens
gaactgaaagtattacttatttcaaattttagtgaggaaatgggcaaggaactataaaatgtaaaatatttaaaacttttttttatgtctatacccttattccatccccctgtacactacaagtcagaaatttcttaggcattcagtttccattagaaactggtctgtaaccttctacgtgatgtttctcttagagtgaaragacctttacctacactgtggtgcaccagagtttaaagttggagagttcaattatatgaaatttaactgtaccctgtagatttcgagagatacgttttaatatcagttggtaggatgtaataccacgtttgcggcagtagtccctctagtactattctccatatacttcctatgtaaacctagtaggagtgtatttccccacatctctgaagttagatttggccaatgccatttctagtgaaatgtgaaatagatatgtctgtcgcttccagggaaagccattaagagctggtgtacaatttacaaaatttattttcccctactctggacccagtgacattcacaatggtggagaatatttttgcctgcatccttgagaaaggacaaatgtggaacaaagcttccatctcattcttattactgttaaaaatataaaagaaatatagttttaagccactgagattttggtgatttttgtcaccaccacataatctagccta